(19) United States
(12) Patent Application Publication
(10) Pub. No.: US 2026/0337156 A1
(43) Pub. Date: Oct. 29, 2026

(54) SOLUBILIZING COMPOUNDS

(71) Applicant: Vivesto AB, Uppsala (SE)

(72) Inventors: Karl Mattias ANDERSSON, Uppsala (SE); Khalil CHAJARA, Uppsala (SE); Roger Wilhelm SOHLBERG, Uppsala (SE); Samaneh PAZESH, Uppsala (SE); Kai WILKINSON, Uppsala (SE); Charles DEMMER, Uppsala (SE)

(21) Appl. No.: 19/115,553

(22) PCT Filed: Sep. 25, 2023
(86) PCT No.: PCT/EP2023/076408

§ 371 Date: Mar. 26, 2025

(30) Foreign Application Priority Data
Sep. 26, 2022 (SE) 2251107-5

(51) Int. Cl. C07C 309/51; A61K 9/107; A61K 31/136; A61K 31/337; A61K 31/704; C07D 295/192

(52) U.S. Cl. CPC C07C 309/51; C07D 295/192; A61K 9/1075; A61K 31/136; A61K 31/337; A61K 31/704; C07C 2601/14

(57) ABSTRACT

The present invention relates to tertiary amide compounds of formula (II), or a pharmaceutically acceptable salt thereof, wherein R⁷ comprises a group X which is —S(═O)₂OH, —S(═O)OH or —P(═O)(OH)₂— The compounds are capable of solubilizing otherwise insoluble drugs. By enhancing the solubility of poorly soluble or insoluble drugs, e.g. hydrophobic drugs, the compounds of the invention make it possible to manufacture formulations of such drugs. The invention also relates to a pharmaceutical composition comprising a compound of formula (I), to a drug micelle comprising a compound of formula (I) and a drug, and to the use of such pharmaceutical composition or drug micelle in the treatment of cancer.

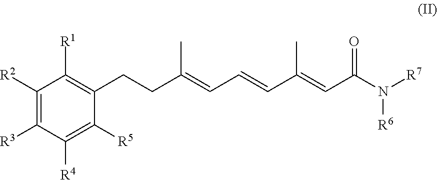

### TECHNICAL FIELD

The present invention relates to tertiary amide compounds of formula (I), or a pharmaceutically acceptable salt thereof. The compounds are capable of solubilizing otherwise insoluble drugs. By enhancing the solubility of poorly soluble or insoluble drugs, e.g. hydrophobic drugs, the compounds of the invention make it possible to manufacture formulations of such drugs. The invention also relates to a pharmaceutical composition comprising a compound of formula (I), to a drug micelle comprising a compound of formula (I) and a drug, and to the use of such pharmaceutical composition or drug micelle in the treatment of cancer.

### BACKGROUND TO THE INVENTION

Many important pharmaceuticals are poorly water soluble. Classical examples include the cytostatic compounds in the taxane group (paclitaxel, docetaxel and cabazitaxel), where the lack of solubility complicates the intravenous administration.

Multiple solutions to the solubility problem have been disclosed in the literature, but none are without drawbacks. A good solubilizing agent should be non-toxic, stable, cost-effective and easy to handle. The solubilizing agent may also affect the pharmacokinetics and pharmacodynamics of the active ingredient in ways that can either be desirable or non-desirable, depending on the clinical situation at hand. Ideally, the solubilizing agent enhances the potency of the active ingredient while at the same time minimizes side-effects.

WO 00/47589, WO 02/092600 and WO 2004/009538 disclose retinol derivatives that are able to form micelles and enhance the potency of cytotoxic agents. More recently, WO 2021/008516 disclosed acitretin derivatives capable of encapsulating an insoluble drug, wherein the formed micelles have high drug loading capacity and good stability.

Despite the progress that previously has been made in this field, there is a continued need for further compounds that can solubilize poorly soluble drugs and provide stable formulations of such drugs. It is therefore an object of the present invention to provide additional solubilizing compounds having an optimized profile with respect to desired properties e.g. micelle-forming properties and stability of micellar formulations.

### SUMMARY OF THE PRESENT INVENTION

The inventors have developed retinoyl-derivatives useful e.g. in pharmaceutical formulations with poorly water soluble drugs. Examples 1-10 demonstrate the synthesis and characterization of 10 different compounds within the general formulae described in more detail below. The Examples also characterize pharmaceutical formulations with the new compounds with drugs. Using docetaxel, cabazitaxel and cyclosporin as model compounds, the inventors demonstrate stable micellar formulations of the compounds and the drugs.

The invention is described in detail below. Certain main aspects of the present invention are defined in the appended independent claims. Certain preferred embodiments are set out in the dependent claims.

### DETAILED DESCRIPTION

In a first aspect, the invention relates to a compound of formula (I),

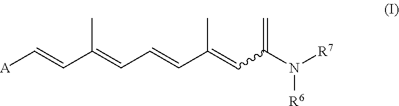

wherein
A is

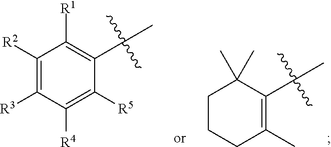

R¹, R², R³, R⁴ and R⁵ are each independently selected from the group consisting of hydrogen, halogen, hydroxy, C₁₋₆ alkyl, C₁₋₆ haloalkyl, C₁₋₆ alkoxy or C₁₋₄ alkoxycarbonyl;
the bond [symbol] is either cis or trans;
R⁶ is selected from the group consisting of halogen, C₁₋₄ alkyl, C₁₋₄ hydroxyalkyl, C₃₋₈ cycloalkyl, C₁₋₄ alkylcarbonyl, C₂₋₄ alkenylcarbonyl, C₃₋₈ cycloalkylcarbonyl, aminocarbonyl-C₁₋₄ alkyl, —C(═NH)NH₂, and phenyl, wherein phenyl is optionally substituted with one or more substituents selected from the group consisting of halogen, hydroxy, C₁₋₄ alkyl, C₁₋₄ alkoxy and amino;
R⁷ is —(CR^{8A}R^{8B})ₙ—X, benzyl-X or C₃₋₇-cycloalkyl-(CR^{8A}R^{8B})ₘ—X, wherein n is an integer 2 or 3 and m is an integer 1 or 2, and wherein benzyl is optionally additionally substituted with one or more substituents selected from the group consisting of halogen, hydroxy, C₁₋₄ alkyl, C₁₋₄ alkoxy and amino;
or R⁶ and R⁷, together with the nitrogen atom to which they are attached, form a 5- to 7-membered saturated heterocyclic ring, which is substituted with —(CR^{8A}R^{8B})ₚ—X, phenyl-X or benzoyl-X, wherein p is an integer 1 or 2, and wherein phenyl or benzoyl is optionally additionally substituted with one or more substituents selected from the group consisting of halogen, hydroxy, C₁₋₄ alkyl, C₁₋₄ alkoxy and amino;
R^{8A} and R^{8B} are each independently selected from the group consisting of hydrogen, hydroxy, C₁₋₄ alkyl, C₁₋₄ alkoxy, —S(═O)₂OH, —S(═O)OH and —P(═O)(OH)₂; and
X is —S(═O)₂OH, —S(═O)OH or —P(═O)(OH)₂;
   or a pharmaceutically acceptable salt thereof.

In a more preferred embodiment, the invention relates to a compound of formula (I)

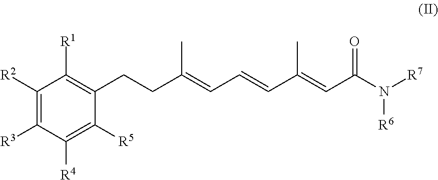

wherein
R¹, R², R³, R⁴ and R⁵ are each independently selected from the group consisting of hydrogen, halogen, hydroxy, C₁₋₆ alkyl, C₁₋₆ haloalkyl, C₁₋₆ alkoxy or C₁₋₄ alkoxycarbonyl;
R⁶ is selected from the group consisting of halogen, C₁₋₄ alkyl, C₁₋₄ hydroxyalkyl, C₃₋₈ cycloalkyl, C₁₋₄ alkylcarbonyl, C₂₋₄ alkenylcarbonyl, C₃₋₈ cycloalkylcarbonyl, aminocarbonyl-C₁₋₄ alkyl, —C(═NH)NH₂, and phenyl, wherein phenyl is optionally substituted with one or more substituents selected from the group consisting of halogen, hydroxy, C₁₋₄ alkyl, C₁₋₄ alkoxy and amino;
R⁷ is —(CR^{8A}R^{8B})ₙ—X, benzyl-X or C₃₋₇-cycloalkyl-(CR^{A}R^{8B})ₘ—X, wherein n is an integer 2 or 3 and m is an integer 1 or 2, and wherein benzyl is optionally additionally substituted with one or more substituents selected from the group consisting of halogen, hydroxy, C₁₋₄ alkyl, C₁₋₄ alkoxy and amino;
or R⁶ and R⁷, together with the nitrogen atom to which they are attached, form a 5- to 7-membered saturated heterocyclic ring, which is substituted with —(CR^{8A}R^{8B})ₚ—X, phenyl-X or benzoyl-X, wherein p is an integer 1 or 2, and wherein phenyl or benzoyl is optionally additionally substituted with one or more substituents selected from the group consisting of halogen, hydroxy, C₁₋₄ alkyl, C₁₋₄ alkoxy and amino;
R^{8A} and R^{8B} are each independently selected from the group consisting of hydrogen, hydroxy, C₁₋₄ alkyl, C₁₋₄ alkoxy, —S(═O)₂OH, —S(═O)OH and —P(═O)(OH)₂; and
X is —S(═O)₂OH, —S(═O)OH or —P(═O)(OH)₂;
   or a pharmaceutically acceptable salt thereof.

In some embodiments, R¹, R², R³, R⁴ and R⁵ are each independently selected from the group consisting of hydrogen, hydroxy, methyl and methoxy. In some embodiments, R¹, R², R³, R⁴ and R⁵ are each independently selected from the group consisting of hydrogen, methyl and methoxy. In some embodiments, R¹, R² and R⁵ are each methyl, R³ is methoxy and R⁴ is hydrogen.

In some embodiments, R⁶ is selected from the group consisting of C₁₋₄ alkyl, C₁₋₄ hydroxyalkyl, C₃₋₈ cycloalkyl, C₁₋₄ alkylcarbonyl, C₂₋₄ alkenylcarbonyl and aminocarbonyl-C₁₋₄ alkyl. In some embodiments, R⁶ is methyl, 2-hydroxyethyl, cyclohexyl, prop-2-enoyl, 1,1-bis(hydroxymethyl)-2-hydroxyethyl or aminocarbonylmethyl. In some embodiments, R⁶ is methyl, 2-hydroxyethyl, cyclohexyl or aminocarbonylmethyl. In some embodiments, R⁶ is methyl. In some embodiments, R⁶ is cyclohexyl.

In some embodiments, R⁷ is —(CR^{8A}R^{8B})ₙ—X, wherein n is an integer 2 or 3 and wherein R^{8A} and R^{8B} are each independently selected from the group consisting of hydrogen, hydroxy, C₁₋₄ alkyl and C₁₋₄ alkoxy. In a more preferred embodiment, R⁷ is —(CR^{8A}R^{8B})ₙ—X, wherein n is an integer 2 or 3 and wherein R^{8A} and R^{8B} are each independently selected from the group consisting of hydrogen and hydroxy.

In some embodiments, R⁷ is benzyl-X, wherein benzyl is optionally additionally substituted with one or more substituents selected from the group consisting of halogen, hydroxy and amino. In a more preferred embodiment, R⁷ is benzyl-X, wherein benzyl is additionally substituted with amino.

In some embodiments, R⁷ is C₃₋₇-cycloalkyl-(CR^{A}R^{8B})ₘ—X, wherein m is an integer 1 or 2, and wherein R^{8A} and R^{8B} are each independently selected from the group consisting of hydrogen and hydroxy. In a more preferred embodiment, R⁷ is cyclohexyl-methyl-X.

In some embodiments, R⁶ and R⁷, together with the nitrogen atom to which they are attached, form a 6-membered saturated heterocyclic ring, which is substituted with —(CR^{8A}R^{8B})ₚ—X, phenyl-X or benzoyl-X, wherein p is an integer 1 or 2. In a more preferred embodiment, R⁶ and R⁷, together with the nitrogen atom to which they are attached, form a piperidine or piperazine ring, which is substituted with ethyl-X, phenyl-X or benzoyl-X.

In some embodiments, X is —S(═O)₂OH.

In a more preferred embodiment, the invention relates to a compound of formula (II), wherein
R¹, R², R³, R⁴ and R⁵ are each independently selected from the group consisting of hydrogen, methyl and methoxy;
R⁶ is methyl, 2-hydroxyethyl, cyclohexyl or aminocarbonylmethyl; and
X is —S(═O)₂OH;
   or a pharmaceutically acceptable salt thereof.

In some embodiments, the invention relates to a compound of formula (III),

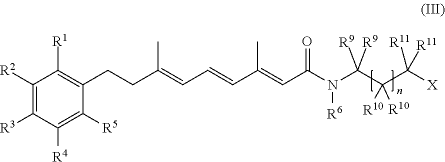

wherein
R¹, R², R³, R⁴ and R⁵ are each independently selected from the group consisting of hydrogen, halogen, C₁₋₆ alkyl, C₁₋₆ haloalkyl, C₁₋₆ alkoxy or C(═O)OR¹⁰; wherein R¹⁰ is C₁₋₄ alkyl;
R⁶ is selected from the group consisting of halogen, C₁₋₄ alkyl, C₁₋₄ hydroxyalkyl, C₃₋₈ cycloalkyl, C₁₋₄ alkylcarbonyl, C₂₋₄ alkenylcarbonyl, C₃₋₈ cycloalkylcarbonyl, —C(═NH)NH₂, and phenyl, wherein phenyl is optionally substituted with one or more substituents selected from the group consisting of halogen, hydroxy, C₁₋₄ alkyl, C₁₋₄ alkoxy and amino;
Each R⁹ is independently selected from the group consisting of hydrogen, hydroxy, C₁₋₄ alkyl, C₁₋₄ alkoxy, —S(═O)₂OH, —S(═O)OH and —P(═O)(OH)₂, or R⁶ and one of R⁹, together with the atoms to which they are attached, form a piperidine ring;
Each R¹⁰ is independently selected from the group consisting of hydrogen, hydroxy, C₁₋₄ alkyl, C₁₋₄ alkoxy, —S(═O)₂OH, —S(═O)OH and —P(═O)(OH)₂;
Each R¹¹ is independently selected from the group consisting of hydrogen, hydroxy, C₁₋₄ alkyl, C₁₋₄ alkoxy, —S(═O)₂OH, S(═O)OH and —P(═O)(OH)₂;
n is an integer 0 or 1; and
X is —S(═O)₂OH, —S(═O)OH or —P(═O)(OH)₂;
   or a pharmaceutically acceptable salt thereof.

In some embodiments, the invention relates to a compound of formula (III),

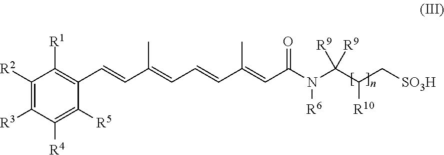

wherein
R¹, R², R³, R⁴ and R⁵ are each independently selected from the group consisting of hydrogen, methyl and methoxy;
R⁶ is methyl, cyclohexyl, prop-2-enoyl or 1,1-bis(hydroxymethyl)-2-hydroxyethyl;
Each R⁹ is independently selected from the group consisting of hydrogen and methyl;
R¹⁰ is hydrogen or hydroxy; and
n is an integer 0 or 1;
   or a pharmaceutically acceptable salt thereof.

In some embodiments of formula (III), R¹, R² and R⁵ are each methyl, R³ is methoxy and R⁴ is hydrogen.

In a particular embodiment, the invention relates to a compound selected from the group consisting of:

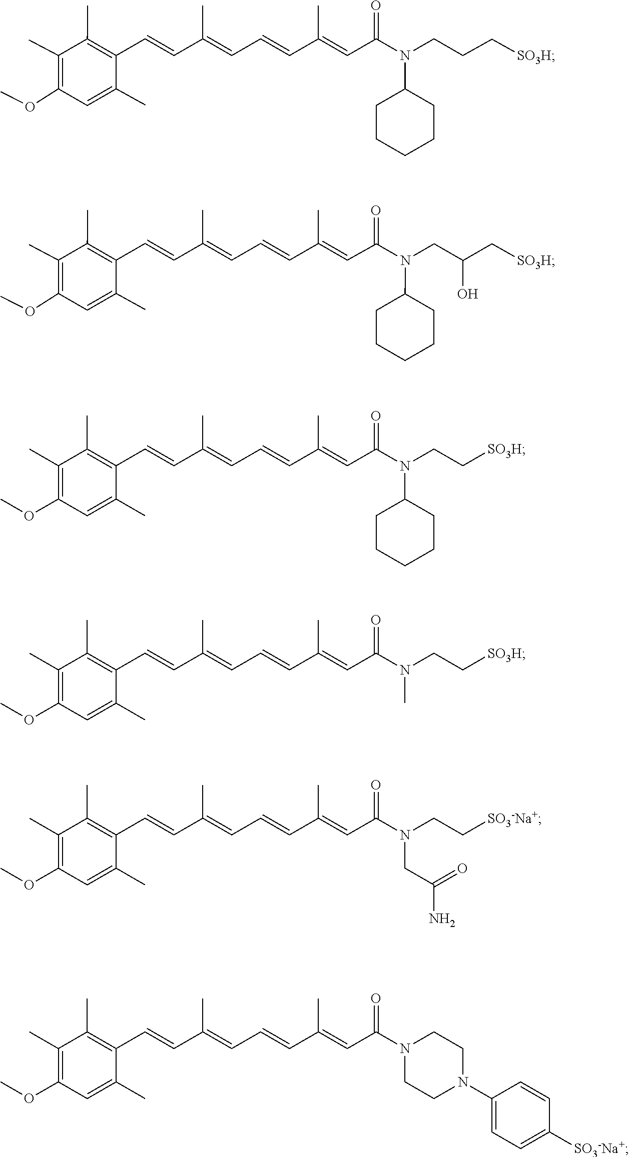

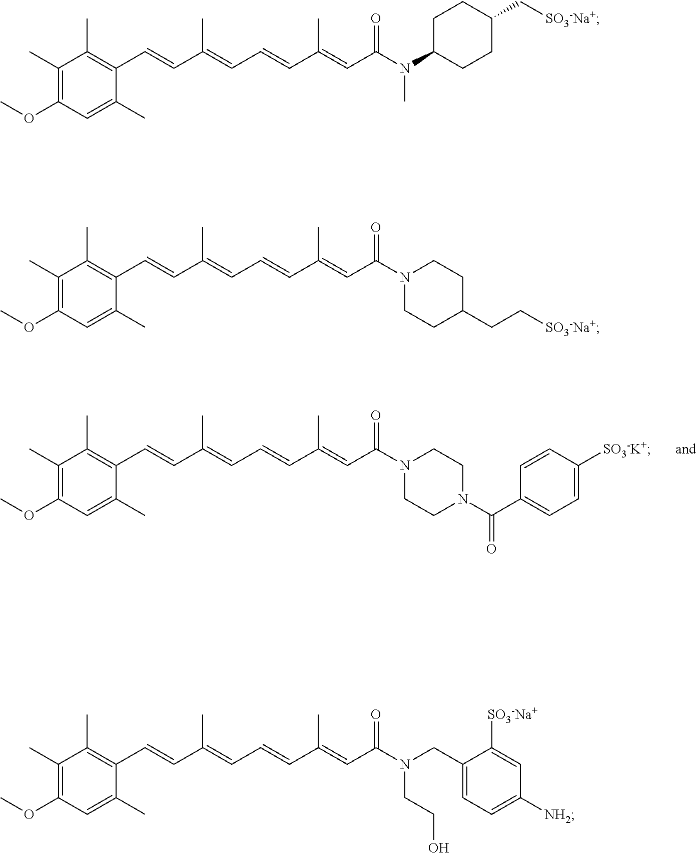

or a pharmaceutically acceptable salt thereof.

As used herein, the term “halo” refers to fluoro, chloro, bromo and iodo.

As used herein, the term “C₁₋₆ alkyl” refers to a straight or branched alkyl group having from 1 to 6 carbon atoms, and the term “C₁₋₄ alkyl” refers to a straight or branched alkyl group having from 1 to 4 carbon atoms. Examples of C₁₋₄ alkyl include methyl, ethyl, n-propyl, isopropyl, n-butyl, isobutyl, sec-butyl and tert-butyl.

As used herein, the term “C₂₋₄ alkenyl” refers to a straight or branched alkenyl group having from 2 to 4 carbon atoms and at least one double bond. Examples of C₂₋₄ alkenyl include ethenyl (vinyl), allyl and 1,3-butadienyl.

As used herein, the term “C₁₋₆ haloalkyl” refers to a straight or branched C₁₋₆ alkyl group, as defined herein, wherein one or more hydrogen atoms have been replaced with halogen. Examples of C₁₋₆ haloalkyl include chloromethyl, fluoroethyl and trifluoromethyl.

As used herein, the term “C₁₋₄ hydroxyalkyl” refers to a straight or branched C₁₋₄ alkyl group, as defined herein, wherein one or more hydrogen atoms have been replaced with a hydroxy group (—OH). Examples of C₁₋₄ hydroxyalkyl include hydroxymethyl, 2-hydroxyethyl and 1,1-bis(hydroxymethyl)-2-hydroxyethyl.

As used herein, the terms “C₁₋₄ alkoxy” refers to a straight or branched C₁₋₄ alkyl group attached to the remainder of the molecule through an oxygen atom.

As used herein, the term “C₃₋₈ cycloalkyl” refers to a monocyclic saturated hydrocarbon ring having from 3 to 8 carbon atoms. Examples of C₃₋₈ cycloalkyl include cyclopropyl, cyclobutyl, cyclopentyl and cyclohexyl.

As used herein, the term “C₁₋₄ alkylcarbonyl” refers to a straight or branched C₁₋₄ alkyl group, as defined herein, bound to a carbonyl group. The terms “C₂₋₄ alkenylcarbonyl” and “C₃₋₈ cycloalkylcarbonyl” should be construed accordingly. Examples of C₁₋₄ alkylcarbonyl include ethylcarbonyl and tert-butylcarbonyl. Examples of C₂₋₄ alkenylcarbonyl include prop-2-enoyl (acryloyl). Examples of C₃₋₈ cycloalkylcarbonyl include cyclopropylcarbonyl and cyclohexylcarbonyl.

A suitable pharmaceutically acceptable salt of a compound of the invention is, for example, a base-addition salt of a compound of the invention, such as an alkali metal salt (e.g., a sodium or potassium salt); an alkaline earth metal salt (e.g., a calcium or magnesium salt); an ammonium salt; a basic amino acid (e.g., arginine, lysine, histidine); a betaine; or a salt with an organic base which affords a physiologically acceptable cation, for example a salt with methylamine, dimethylamine, a trialkylamine (e.g., trimethylamine), piperidine, pyrrolidine, morpholine, choline, an ethanolamine, tris(hydroxymethyl)aminomethane (TRIS) or triethanolamine (tris-(2-hydroxyethyl)amine).

### Pharmaceutical Compositions

Unless explicitly mentioned otherwise, a reference to compounds of formula (I) herein should be understood to also include compounds of formulas (II) and (Ill).

In another aspect, the invention relates to a pharmaceutical composition comprising a compound according to formula (I), or a pharmaceutically acceptable salt thereof. The pharmaceutical composition optionally also comprises one or more pharmaceutically acceptable excipients. The excipients may e.g. include water, aqueous buffers, saline, co-solvents, fillers, binders, disintegrants, glidants and lubricants. In general, pharmaceutical compositions may be prepared in a conventional manner using conventional excipients.

Examples of suitable co-solvents include, but are not limited to, ethanol, propylene glycol and polyethylene glycol (such as PEG 400).

Examples of suitable fillers include, but are not limited to, dicalcium phosphate dihydrate, calcium sulfate, lactose (such as lactose monohydrate), sucrose, mannitol, sorbitol, cellulose, microcrystalline cellulose, dry starch, hydrolyzed starches and pregelatinized starch.

Examples of suitable binders include, but are not limited to, starch, pregelatinized starch, gelatin, sugars (such as sucrose, glucose, dextrose, lactose and sorbitol), polyethylene glycol, waxes, natural and synthetic gums (such as acacia gum, xanthan gum, tragacanth gum and carrageenan), sodium alginate, cellulose derivatives (such as hydroxypropylmethylcellulose (or hypromellose), hydroxypropylcellulose and ethylcellulose) and synthetic polymers (such as acrylic acid and methacrylic acid copolymers, methacrylic acid copolymers, methyl methacrylate copolymers, aminoalkyl methacrylate copolymers, polyacrylic acid/polymethacrylic acid copolymers and polyvinylpyrrolidone (povidone)).

Examples of suitable disintegrants include, but are not limited to, dry starch, modified starch (such as (partially) pregelatinized starch, sodium starch glycolate and sodium carboxymethyl starch), alginic acid, cellulose derivatives (such as sodium carboxymethylcellulose, hydroxypropyl cellulose, and low substituted hydroxypropyl cellulose (L-HPC)) and cross-linked polymers (such as carmellose, croscarmellose sodium, carmellose calcium and cross-linked PVP (crospovidone)).

Examples of suitable glidants and lubricants include, but are not limited to, talc, magnesium stearate, calcium stearate, stearic acid, glyceryl behenate, colloidal silica, aqueous silicon dioxide, synthetic magnesium silicate, fine granulated silicon oxide, starch, sodium lauryl sulfate, boric acid, magnesium oxide, waxes (such as carnauba wax), hydrogenated oil, polyethylene glycol, sodium benzoate, polyethylene glycol, and mineral oil.

The pharmaceutical composition may be in a form that is suitable for oral administration, for parenteral administration (including intracutaneous, intradermal, intravenous, subcutaneous, intramuscular, intraperitoneal and intravascular injection or infusion), for topical administration, for ophthalmic administration, for oral administration (including sublingual administration), for nasal administration (e.g., inhalation) or for rectal administration. In a preferred embodiment, the pharmaceutical composition is in the form of liquid formulation that is suitable for oral or parenteral administration. In another embodiment, the pharmaceutical composition is in the form of a solid formulation that is suitable for oral administration, such as a tablet or a capsule. In yet another embodiment, the pharmaceutical composition is in the form of a formulation that is suitable for topical administration, such as an ointment or a cream.

In some embodiments, the pharmaceutical composition further comprises at least one active pharmaceutical ingredient (herein also referred to as “drug” or “API”). In some embodiments, the composition contains more than one active pharmaceutical ingredient.

The active pharmaceutical ingredient may be a small molecule, a macromolecule, a peptide, a protein (such as an enzyme), a nucleic acid, an antigen, an antibody, or a viral vector. As shown in the experimental section, the compounds of the invention form micelles, which may encapsulate the active pharmaceutical ingredient or contain the active pharmaceutical ingredient in the micelle structure. This enhances the solubility of an otherwise poorly soluble or insoluble drug, and makes it possible to manufacture pharmaceutical formulations comprising a poorly soluble or insoluble drug. Such formulation of a poorly soluble or insoluble drug also allows the drug to be administered in a liquid formulation.

Additionally, such formulation may increase the bioavailability of the drug.

A pharmaceutical composition comprising an active pharmaceutical ingredient (such as a poorly soluble or insoluble drug) and at least one compound of formula (I) may further exhibit enhanced pharmacological activity and/or improved therapeutic efficacy. Such a composition may also enhance the distribution of the drug to the target tissue(s), and may additionally lead to fewer undesired side effects. Such compositions may also improve pharmacokinetic properties of the active pharmaceutical ingredient including the elimination half-life, the maximal plasma concentration, clearance, volume of distribution and/or mean residence time.

In some embodiments, the active pharmaceutical ingredient is a hydrophobic drug. Such drugs may e.g. be characterized by having a water solubility at 20 °C of less than 100 μg/mL.

In some embodiments, the active pharmaceutical ingredient is a macrocyclic drug, e.g. a macrolide, a macrocyclic peptide or a metallo-supramolecular compound, such as cyclosporin, rifamycin, rapamycin, vancomycin, dactinomycin, amphotericin B, ivermectin, simeprevir, ixabepilone, sirolimus or tacrolimus.

In some embodiments, the active pharmaceutical ingredient is a cytotoxic drug, e.g. a taxane such as docetaxel, paclitaxel or cabazitaxel; an anthracycline such as aclarubicin, amrubicin, daunorubicin, doxorubicin, epirubicin, idarubicin, pirarubicin, valrubicin or zorubicin; an anthracenedione such as mitoxantrone, losoxantrone, pixantrone, amsacrine, or bisantrene; or a vinca alkaloid such as vinblastine, vincristine, vindesine, vinflunine or vinorelbine.

Also provided herein is a drug micelle, comprising at least one compound of formula (I), or a pharmaceutically acceptable salt thereof, and a drug. In some embodiments, the drug is a hydrophobic drug. In some embodiments, the drug is a cytotoxic drug. In some embodiments, the cytotoxic drug is selected from the group consisting of docetaxel, paclitaxel, cabazitaxel, doxorubicin and mitoxantrone.

In some embodiments, the compound of formula (I) and the drug are present in the composition at a ratio of from about 20:1 to about 1:20 (w/w), such as from about 15:1 to about 1:15, or such as from about 10:1 to about 1:10. In some embodiments, the compound of formula (I) and the hydrophobic drug are present in the micelle in a ratio of about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9 or about 1:10 (w/w).

The particle size of the micelles in the composition is typically in the range of about 5 to about 150 nm. The particle size may be determined e.g. using Dynamic Light Scattering (DLS) in 0.9% NaCl at 1 mg/mL drug concentration, preferably using a red laser with a wavelength of 633 nm.

It has been found that the particle size is dependent on the nature of the compound of formula (I) and the nature of the drug, on the ratio between the compound of formula (I) and the drug, and on the concentration of the compounds in the composition. By varying these conditions, it is possible to tailor the particle size of the micelles, as desired. Depending on circumstances, the micelles may be in equilibrium with “free” compound of formula (I), or a pharmaceutically acceptable salt thereof, and/or with “free” drug. Alternatively, the micelles may reshape over time. The particle size (and consequently the polydispersity index) may therefore change somewhat over time, such as after 4 hours, after 8 hours, after 12 hours or after 24 hours following formation of the micelles. In some embodiments, the particle size is about 5 to about 125 nm, such as about 25 to about 125 nm, about 50 to about 125 nm, about 75 to about 125 nm or about 100 to 125 nm; such as about 25 to about 100 nm, about 25 to about 75 nm or about 25 to about 50 nm; or such as about 50 to 100 nm, about 50 to about 75 nm or about 75 to 100 nm. Preferably, the particle size is less than 100 nm. Preferably, the polydispersity index is less than 0.5, more preferably less than 0.4 and even more preferably less than 0.3. In some embodiments, the polydispersity index is between 0 and 0.5, such as between 0 and 0.4, or such as between 0 and 0.3. Preferably, the particle size distribution is substantially monomodal.

### Methods and Uses

In another aspect, the invention relates to a pharmaceutical composition comprising at least one compound of formula (I), or a pharmaceutically acceptable salt thereof, and a drug, for use in therapy.

In another aspect, the invention relates to a pharmaceutical composition comprising at least one compound of formula (I), or a pharmaceutically acceptable salt thereof, and a cytotoxic drug, for use in the treatment of cancer.

In another aspect, the invention relates to a cytotoxic drug micelle comprising at least one compound of formula (I), or a pharmaceutically acceptable salt thereof, and a cytotoxic drug, for use in the treatment of cancer.

The invention also relates to the use of said pharmaceutical composition or said cytotoxic drug micelle in the manufacture of a medicament for the treatment of cancer. The invention also relates to a method of treating cancer in a subject, such as a human, comprising administering to the subject in need of such treatment a therapeutically effective amount of said pharmaceutical composition or said cytotoxic drug micelle.

Also provided herein is a method for potentiating the efficacy of a pharmaceutically active substance, wherein said substance is prepared in micellar form with at least one compound of formula (I), or a pharmaceutically acceptable salt thereof.

Also provided herein is a method for increasing the solubility of a pharmaceutically active substance, wherein said substance is prepared in micellar form with at least one compound of formula (I), or a pharmaceutically acceptable salt thereof.

Also provided herein is a method for improving the pharmacokinetic or pharmacodynamic properties of a pharmaceutically active substance, wherein said substance is prepared in micellar form with at least one compound of formula (I), or a pharmaceutically acceptable salt thereof.

Also provided herein is a method for improving the storage properties of a pharmaceutically active substance, wherein said substance is prepared in micellar form with at least one compound of formula (I), or a pharmaceutically acceptable salt thereof.

### Radioisotopes

In another aspect, the invention also relates to a compound of formula (I), (II) or (III), as defined herein, yet wherein the compound contains at least one atom of a halogen radioisotope (radioactive isotope).

Preferably, at least one of R¹, R², R³, R⁴ and R⁵ is a halogen radioisotope (radioactive isotope). In some embodiments, one, two, three or four of R¹, R², R³, R⁴ and R⁵ are a halogen radioisotope, and the other are each independently selected from the group consisting of hydrogen, C₁₋₆ alkyl, C₁₋₆ haloalkyl, C₁₋₆ alkoxy or C(═O)OR¹⁰; wherein R¹⁰ is C₁₋₄ alkyl. In some embodiments, one, two, three or four of R¹, R², R³, R⁴ and R⁵ are a halogen radioisotope, and the other are each independently selected from the group consisting of hydrogen, methyl and methoxy. In some embodiments, all of R¹, R², R³, R⁴ and R⁵ are a halogen radioisotope. Particular examples of halogen radioisotopes include fluorine-18, iodine-123, iodine-125 and iodine-131.

Radioisotope-labelled compounds of formula (I) may be useful as radiopharmaceuticals, either for diagnostic or therapeutic purposes.

### DEFINITIONS

As used herein, the term “pharmaceutically acceptable” refers to those compounds, materials, compositions and/or dosage forms that are suitable for human pharmaceutical use and that are generally safe, non-toxic and neither biologically nor otherwise undesirable.

As used herein, the terms “treatment”, “treat” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “comprising” is to be interpreted as including, but not being limited to. All references are hereby incorporated by reference. The arrangement of the present disclosure into sections with headings and subheadings is merely to improve legibility and is not to be interpreted limiting in any way. In particular, the division does not in any way preclude or limit combining features under different headings and subheadings with each other.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

### EXAMPLES

The invention will now be described by the following examples which do not limit the invention in any respect.

### ABBREVIATIONS

API Active Pharmaceutical Ingredient
Boc tert-butyloxycarbonyl
CAPS 3-(cyclohexylamino)-1-propanesulfonic acid
CAPSO 3-(cyclohexylamino)-2-hydroxy-1-propanesulfonic acid
CHES 2-(cyclohexylamino)ethanesulfonic acid
DCM dichloromethane
DIPEA N,N-diisopropylethylamine
DLS dynamic light scattering
DMF dimethylformamide
MTBE methyl tert-butyl ether
PyBOP benzotriazol-1-yloxy-tripyrrolidinophosphonium hexafluorophosphate
RSD relative standard deviation
SDS sodium dodecyl sulfate
TEA triethylamine
TFA trifluoroacetic acid
THF tetrahydrofuran
TLC thin layer chromatography

### EXPERIMENTAL METHODS

Reagents and solvents were purchased from Sigma-Aldrich (Merck) or Biosynth. Room temperature refers to 20-25 °C. Solvent mixture compositions are given as volume percentages or volume ratios.

¹H NMR spectra were recorded at 500 MHz using a Variant Unity-500 spectrometer, or at 300 MHz using a Bruker spectrometer. DMSO-d₆ was used as solvent.

Mass Spectrometer readings were recorded using Dionex UHPLC Ultimate 3000 with DAD detector/Thermo Scientific ISQ EC-Mass Spectrometer.

Merck silica gel RP-18 plates were used for thin layer chromatography (TLC) and developed in a solvent system consisting of methanol:water (7:3, v/v).

Micellar size was measured using a ZetaSizer from Malvern Panalytical.

HPLC was performed on a Chromaster HPLC system (Hitachi). Analyses were performed using a Hypurity C18 column, 250×4.6 mm, 3.0 μm (Thermo Scientific). Mobile phase: solvent A: 10% NH₄OAc (aq), 10% SDS (aq) and 80% acetonitrile, solvent B: 10% NH₄OAc (aq), 10% SDS (aq) and 80% water. Flow rate: 1 mL/min. Run time: 65 min. Pump program:

| Time (min) | Solvent A % | Solvent B % |
|---|---|---|
| 0 | 42.5 | 57.5 |
| 45 | 82 | 18 |
| 46 | 42.5 | 57.5 |
| 65 | 42.5 | 57.5 |

### Example 1

### 1.1 Synthesis of Compound 1

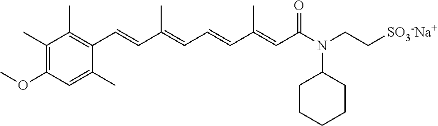

In a 25 mL two-necked flask, acitretin (250 mg, 0.77 mmol) and TEA (92 μL, 0.91 mmol) were dissolved in anhydrous THF (10 mL) and the mixture was cooled to 5-10 °C. A solution of isobutylchloroformate (129 μL, 0.99 mmol) in THF (0.5 mL) was added dropwise to the chilled solution. The resulting mixture was stirred for 30 minutes at 5-10 °C under inert conditions and protected from light.

A solution of CHES (222 mg, 1.07 mmol) and TEA (149 μL, 1.07 mmol) in methanol (3 mL) was added to the reaction mixture and the resulting solution was stirred under inert conditions at room temperature for 2.5 hours. Acetic acid (200 μL) was then carefully added to the reaction mixture. The solution was evaporated to dryness and the resulting crude was extracted with MTBE (20 mL) and water (25 mL). The layers were separated and the organic layer was discarded. Sodium hydrogencarbonate (300 mg) was added carefully to the aqueous layer. When effervescence had stopped, brine (25% NaCl-solution, 20 mL) was added and the resulting aqueous solution was extracted with ethyl acetate (20 mL). The layers were separated and the aqueous was phase discarded. The organic layer was washed with a mixture of brine (12.5% NaCl-solution, 20 mL) and methanol (2 mL). The aqueous layers were discarded, and the organic layer was evaporated under reduced pressure (40-70 mbar at 39 °C). The release of the vacuum was made with inert gas (N₂). The product was washed with MTBE (10 mL) and dried under vacuum. The obtained product was purified by column chromatography (RP-C18; eluent: MeOH/water 7:3) to afford the product as a yellow solid (purity 94.8%). Yield: 50%.

¹H NMR (500 MHz, DMSO-d₆): δ 6.85 (ddd, 1H, J=30.2, 15.2, 11.4 Hz, CH═CH—); 6.67 (s, 1H, Ph-H); 6.65 (d, 1H, J=16.4 Hz, —CH═CH—); 6.42 (dd, 1H, J=16.4 Hz, —CH═CH—); 6.31 (s, 1H, ═C—CH); 6.25-6.19 (m, 2H, —CH═CH—); 3.78 (s, 3H, —O—CH₃); 3.45 (m, 2H, CH₂); 3.35 (m, 2H, CH₂); 2.65 (m, 1H, CH cyclohexyl); 2.50 (s, 3H, CH₃); 2.23 (s, 3H, CH₃); 2.21 (s, 3H, CH₃); 2.12 (s, 6H, CH₃); 1.9 (s, 3H, CH₃); 1.77, (m, a (2H) cyclohexyl); 1.62, (m, e (2H) cyclohexyl); 1.28, (m, a (2H) cyclohexyl); 1.10, (m, e (2H) cyclohexyl); 1.05, (m, 2H, cyclohexyl).

m/z=538.26; 516.27.

### 1.2. Formulation with API

1.2.1 Formulation with Docetaxel

3.8 mg of compound 1 was dispensed into a 25 mL round bottom flask and dissolved in methanol (2 mL). 240 μL of a stock solution of docetaxel in methanol (5 mg/mL) was added.

The solution was evaporated to dryness in a rotary evaporator. Final drying was performed in a desiccator to remove residual solvent. Hydration of the dried film was performed by adding water for injection (1.2 mL). Finally, 0.9% sodium chloride solution (1.0 mL) was added and a resulting yellow transparent solution was obtained. Drug loading capacity is defined as the ratio of amount API:(API+excipient).

| Docetaxel | Compound 1 | Particle size | Polydispersity | Drug loading |
|---|---|---|---|---|
| [mg] | [mg] | [nm] | index | capacity [%] |
| 1.2 | 3.8 | 7.0 | 0.20 | 24 |

20 mg of example 1 was dispensed into a 25 mL round bottom flask and dissolved in methanol (3 mL). 800 μL of a stock solution of docetaxel in methanol (5 mg/mL) was added. The solution was evaporated to dryness in a rotary evaporator. Final drying was performed in a desiccator to remove residual solvent. Hydration of the dried film was performed by adding water for injection (1.0 mL) to a concentration of 4 mg/mL of docetaxel. The 4 mg/mL concentration was further diluted with 0.9% sodium chloride according to the table below.

### Ratio 5:1

| Docetaxel conc. | Particle size | Polydispersity | Drug loading |
|---|---|---|---|
| [mg/mL] | [nm] | index | capacity [%] |
| 1 | 29.2 | 0.24 | 17 |
| 0.5 | 41.5 | 0.24 | |
| 0.25 | 49.6 | 0.25 | |

11 mg of example 1 was dispensed into a 25 mL round bottom flask and dissolved in methanol (2 mL). 740 μL of a stock solution of docetaxel in methanol (4.96 mg/mL) was added. The solution was evaporated to dryness in a rotary evaporator. Final drying was performed in a desiccator to remove residual solvent. Hydration of the dried film was performed by adding sodium chloride 0.9% to a concentration of 1 mg/mL docetaxel resulting in a yellow transparent solution.

### Ratio 3:1

| Docetaxel conc. | Particle size | Polydispersity | Drug loading |
|---|---|---|---|
| [mg/mL] | [nm] | index | capacity [%] |
| 1 | 47.7 | 0.24 | 25 |
| 0.5 | 52.6 | 0.25 | |
| 0.25 | 56.0 | 0.29 | |

A stock solution of docetaxel in methanol was mixed with a stock solution of compound 1 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of docetaxel. The final weight ratio of docetaxel to micelle forming agent was 1:5. Measurements of particle size and polydispersity index were made over time, as shown in the following table. Small increases in the particle size and polydispersity index over time were noted. The changes were within acceptable range.

| Time [h] | Particle size [nm] | Polydispersity index |
|---|---|---|
| 0 | 30.77 | 0.10 |
| 1 | 36.60 | 0.16 |
| 2 | 39.44 | 0.16 |
| 4 | 41.38 | 0.18 |
| 6 | 41.19 | 0.20 |
| 24 | 43.98 | 0.21 |

A serial dilution was performed for the system docetaxel:compound 1. A stock solution of docetaxel in methanol was mixed with a stock solution of the micelle forming agent (compound 1) in a round bottom flask at a weight ratio of 1:5. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution. The micellar size and RSD showed no significant trend during dilution.

| Concentration of | Particle | RSD Particle | Polydispersity | RSD |
|---|---|---|---|---|
| compound 1 [mM] | size [nm] | size | index, pdi | pdi |
| 4.8 | 35.4 | 1.89 | 0.11 | 7.67 |
| 2.4 | 36.2 | 1.15 | 0.14 | 13.67 |
| 1.2 | 36.5 | 0.98 | 0.16 | 4.07 |
| 0.6 | 36.53 | 1.04 | 0.18 | 4.18 |
| 0.3 | 37.85 | 1.93 | 0.25 | 11.76 |
| 0.15 | 34.06 | 1.43 | 0.34 | 7.08 |
| 0.12 | 32.72 | 0.82 | 0.39 | 3.04 |
| 0.08 | 33.73 | 1.19 | 0.45 | 17.20 |

1.2.2 Formulation with Cabazitaxel

4.5 mg of compound 1 was dispensed into a 25 mL round bottom flask and dissolved in methanol (2 mL). 1500 μL of a stock solution of cabazitaxel in methanol (1.00 mg/mL) was added. The solution was evaporated to dryness in a rotary evaporator. Final drying was performed in a desiccator to remove residual solvent. Hydration of the dried film was performed by adding sodium chloride 0.9% to a concentration of 1 mg/mL cabazitaxel resulting in a yellow transparent solution. The results for different dilutions are shown in the table below.

### Ratio 3:1

| Cabazitaxel conc. | Particle size | Polydispersity | Drug loading |
|---|---|---|---|
| [mg/mL] | [nm] | index | capacity [%] |
| 1 | 87.6 | 0.145 | 25 |
| 0.5 | 111.2 | 0.102 | 25 |
| 0.25 | 121.2 | 0.079 | 25 |

1.2.3 Formulation with Cyclosporin

4.5 mg of compound 1 was dispensed into a 25 mL round bottom flask and dissolved in methanol (2 mL). 1500 μL of a stock solution of cyclosporin in methanol (1.00 mg/mL) was added. The solution was evaporated to dryness in a rotary evaporator. Final drying was performed in a desiccator to remove residual solvent. Hydration of the dried film was performed by adding sodium chloride 0.9% to a concentration of 1 mg/mL cyclosporin resulting in a yellow transparent solution. The results for different dilutions are shown in the table below.

### Ratio 3:1

| Cyclosporin conc. | Particle size | Polydispersity | Drug loading |
|---|---|---|---|
| [mg/mL] | [nm] | index | capacity [%] |
| 1 | 41.7 | 0.212 | 25 |
| 0.5 | 53.3 | 0.293 | 25 |
| 0.25 | 60.1 | 0.258 | 25 |

A stock solution of cyclosporin in methanol was mixed with a stock solution of compound 1 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of cyclosporin. The resulting solution was filtered through a 0.22 μm sterile filter and further diluted with 0.9% sodium chloride solution to 0.5 mg/mL of cyclosporin. The final weight ratio of cyclosporin to micelle forming agent was 1:3. Measurements of particle size and polydispersity index were made over time, as shown in the following table. Small increases in the particle size and variations of polydispersity index over time were noted. The changes were within acceptable range.

| Time | Particle | Polydispersity |
|---|---|---|
| [h] | size [nm] | index |
| 0 | 30.71 | 0.10 |
| 1 | 44.70 | 0.22 |
| 2 | 41.29 | 0.10 |
| 4 | 44.53 | 0.10 |
| 6 | 49.61 | 0.19 |
| 24 | 51.37 | 0.14 |

### Example 2

### 2.1 Synthesis of Compound 2

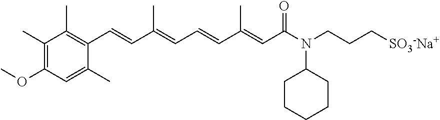

In a 25 mL two-necked flask, acitretin (500 mg, 1.5 mmol) and TEA (216 μL, 1.68 mmol) were dissolved in anhydrous THF (10 mL), and the mixture was cooled to 5-10 °C. A solution of isobutylchloroformate (260 μL, 1.97 mmol) in THF (0.5 mL) was added dropwise to the chilled solution. The resulting mixture was stirred for 30 min at 5-10 °C under inert conditions and protected from light.

A solution of CAPS (475 mg, 2.14 mmol) and TEA (276 μL, 2.14 mmol) in methanol (3 mL) was added to the reaction mixture and the resulting solution was stirred under inert conditions at room temperature for 2 hours. Acetic acid (600 μL) was then carefully added to the reaction mixture. The solution was evaporated to dryness and the resulting crude was extracted with MTBE (20 mL) and water (25 mL). The layers were separated, and the organic layer was discarded. Sodium hydrogencarbonate (600 mg) was added carefully to the aqueous layer. When effervescence had stopped, brine (25% NaCl-solution, 20 mL) was added and the resulting aqueous solution was extracted with ethyl acetate (20 mL). The layers were separated and the aqueous phase was discarded. The organic layer was washed with a mixture of brine (12.5% NaCl-solution, 20 mL) and methanol (1-3 mL). The aqueous layers were discarded, and the organic layer was evaporated under reduced pressure (40-70 mbar at 39 °C). The release of the vacuum is made with inert gas (N₂). The obtained product was purified by column chromatography (RP-C18; eluent: MeOH/water 7:3) to give a yellow product (purity 88.2%). Yield: 10%.

¹H NMR (500 MHz, DMSO-d₆): δ 6.75 (m, 1H, CH═CH—); 6.70 (s, 1H, Ph-H); 6.65 (m, 1H, —CH═CH—); 6.45 (dd, 1H, —CH═CH—); 6.27 (s, 1H, C═CH—); 6.20-6.25 (m, 2H, —CH═CH—); 3.75 (s, 3H, —O—CH₃); 3.40-3.35 (m, 4H, CH₂); 3.48 (m, 2H,); 2.60 (m, (1H) cyclohexyl); 2.26 (s, 3H, CH₃); 2.05 (s, 3H, CH₃); 1.95 (s, 3H, CH₃); 1.75 (s, 6H, CH₃); 1.55 (m, 6H, cyclohexyl); 1.25, (m, 2H, cyclohexyl); 1.14, (m, 2H, cyclohexyl).

### 2.2 Formulation with API

2.2.1 Formulation with Docetaxel

4.0 mg of compound 2 was dispensed into a 25 mL round bottom flask and dissolved in methanol (3 mL). 250 μL of a stock solution of docetaxel in methanol (5 mg/mL) was added. The solution was evaporated to dryness in a rotary evaporator. Final drying was performed in a desiccator to remove residual solvent. Hydration of the dried film was performed by adding water for injection (1.25 mL). This solution was mixed 1:1 v/v with 0.9% sodium chloride solution and a resulting yellow transparent solution was obtained. The results are shown below.

| Docetaxel | Compound 2 | Particle | Polydispersity | Drug loading |
|---|---|---|---|---|
| [mg] | [mg] | size [nm] | index | capacity [%] |
| 1.25 | 4.0 | 17.9 | 0.72 | 24 |

### Example 3

### 3.1 Synthesis of Compound 3

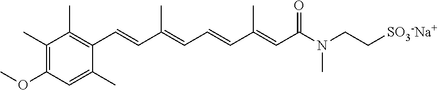

In a 25 mL two-necked flask, acitretin (500 mg, 1.53 mmol) and TEA (236 μL, 1.80 mmol) were dissolved in anhydrous THF (15 mL) and the reaction mixture was cooled to 5-10 °C. A solution of isobutylchloroformate (260 μL, 1.97 mmol) in THF (0.8 mL) was dropwise added to the chilled solution. The resulting mixture was stirred for 30 min at 5-10 °C under inert conditions and protected from light.

A solution of 2-(methylamino)ethane-1-sulfonic acid (298 mg, 2.14 mmol) and TEA (286 μL, 2.17 mmol) in methanol (3 mL) was added to the reaction mixture and the resulting solution was stirred under inert conditions at room temperature for 2.5 hours. Acetic acid (500 μL) was then carefully added to the reaction mixture. The solution was evaporated to dryness and the resulting crude was extracted with MTBE (20 mL) and water (25 mL). The layers were separated and the organic layer was discarded. Sodium hydrogencarbonate (400 mg) was added carefully to the aqueous layer. When effervescence had stopped, brine (25% NaCl-solution, 20 mL) was added and the resulting aqueous solution was extracted with ethyl acetate (20 mL). The layers were separated and the aqueous was phase discarded. The organic layer is washed with a mixture of brine (12.5% NaCl-solution, 20 mL) and methanol (1-3 mL). The aqueous layers are discarded, and the organic layer was evaporated under reduced pressure (40-70 mbar at 39 °C). The release of the vacuum is made with inert gas (N₂). The obtained product was purified by column chromatography (RP-C18; eluent: MeOH/water 7:3) to afford the product as a yellow solid (purity 99.4%). Yield: 55%.

¹H NMR (500 MHz, DMSO-d₆): δ 6.86 (t, 1H, J=13.3 Hz, CH═CH—), 6.72-6.57 (m, 2H); 6.44 (s, 1H, —CH═CH—); 6.32-6.18 (m, 3H, —CH═CH); 3.70 (s, 3H, —O—CH₃); 3.70-3.50 (d, 2H, CH₂); 3.20-2.70 (t, 3H, N—CH₃); 2.70-2.67 (d, CH₂); 2.25 (s, 3H, CH₃); 2.24 (s, 3H, CH₃); 2.07 (s, 9H, CH₃).

m/z=470.19; 448.21.

### 3.2 Formulation with API

3.2.1 Formulation with Docetaxel

4.5 mg of compound 3 was dispensed into a 25 mL round bottom flask and dissolved in methanol (3 mL). 305 μL of a stock solution of docetaxel in methanol (4.96 mg/mL) was added. The solution was evaporated to dryness in a rotary evaporator. Final drying was performed in a desiccator to remove residual solvent. Rehydration of the dried film was performed by adding 0.9% sodium chloride solution (1.5 mL) and a transparent yellow solution was obtained. The results for different dilutions are shown in the table below.

### Ratio 3:1

| Docetaxel | | | |
|---|---|---|---|
| conc. | Particle | Polydispersity | Drug loading |
| [mg/mL] | size [nm] | index | capacity [%] |
| 1 | 9.30 | 0.33 | 25 |
| 0.5 | 11.3 | 0.25 | 25 |
| 0.25 | 11.8 | 0.27 | 25 |

4.5 mg of compound 3 was dispensed into a 25 mL round bottom flask and dissolved in methanol (3 mL). 0.455 μL of a stock solution of docetaxel in methanol (4.96 mg/mL) was added. The solution was evaporated to dryness in a rotary evaporator. Final drying was performed in a desiccator to remove residual solvent. Rehydration of the dried film was performed by adding 0.9% sodium chloride solution (2.25 mL) and a transparent yellow solution was obtained. The results for different dilutions are shown in the table below.

### Ratio 2:1

| Docetaxel | | | |
|---|---|---|---|
| conc. | Particle | Polydispersity | Drug loading |
| [mg/mL] | size [nm] | index | capacity [%] |
| 1 | 13.5 | 0.52 | 25 |
| 0.5 | 19.9 | 0.76 | 25 |
| 0.25 | 14.0 | 0.53 | 25 |

A stock solution of docetaxel in methanol was mixed with a stock solution of compound 3 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of Docetaxel. The final weight ratio of docetaxel to micelle forming agent was 1:3. Measurements of particle size and polydispersity index were made over time, as shown in the following table. Small variations in the particle size and polydispersity index over time were noted. The changes were within acceptable range.

| Time | Particle | Polydispersity |
|---|---|---|
| [h] | size [nm] | index |
| 0 | 6.19 | 0.11 |
| 1 | 7.19 | 0.11 |
| 2 | 10.06 | 0.26 |
| 4 | 10.18 | 0.27 |
| 6 | 10.25 | 0.27 |
| 24 | 6.20 | 0.14 |

3.2.2 Formulation with Cyclosporin

A stock solution of cyclosporin in methanol was mixed with a stock solution of compound 3 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of cyclosporin. The resulting solution was filtered through a 0.22 μm sterile filter and further diluted with 0.9% sodium chloride solution to 0.5 mg/mL of cyclosporin. The final weight ratio of cyclosporin to micelle forming agent was 1:3. Measurements of particle size and polydispersity index were made over time, as shown in the following table. Small variations in the particle size and polydispersity index over time were noted (see table below). The changes were within acceptable range.

| Time | Particle | Polydispersity |
|---|---|---|
| [h] | size [nm] | index |
| 0 | 8.46 | 0.31 |
| 1 | 6.77 | 0.21 |
| 2 | 6.60 | 0.19 |
| 4 | 6.58 | 0.18 |
| 6 | 6.68 | 0.21 |
| 24 | 7.39 | 0.32 |

### Example 4

### 4.1 Synthesis of Compound 4

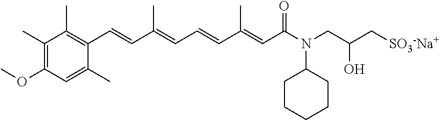

In a 25 mL two-necked flask, acitretin (250 mg, 0.77 mmol) and TEA (95 μL, 0.80 mmol) were dissolved in anhydrous THF (10 mL), and the mixture was cooled to 5-10 °C. A solution of isobutylchloroformate (129 μL, 1.0 mmol) in THF (0.5 mL) was dropwise added to the chilled solution. The resulting mixture was stirred for 30 min at 5-10 °C under inert conditions and protected from light. A solution of CAPSO (260 mg, 1.1 mmol) and TEA (149 μL, 1.16 mmol) in methanol (3 mL) was added to the reaction mixture and the resulting solution was stirred under inert conditions at room temperature for 2 hours. Acetic acid (400 μL) was then carefully added to the reaction mixture. The solution is evaporated to dryness and the resulting crude was extracted with MTBE (20 mL) and water (25 mL). The layers were separated and the organic layer was discarded. Sodium hydrogencarbonate (300 mg) was added carefully to the aqueous layer. When effervescence had stopped, brine (25% NaCl-solution, 20 mL) was added and the resulting aqueous solution was extracted with ethyl acetate (20 mL). The layers were separated and the aqueous phase was discarded. The organic layer was washed with a mixture of brine (12.5% NaCl-solution, 20 mL) and methanol (1-3 mL). The aqueous layers were discarded, and the organic layer was evaporated under reduced pressure (40-70 mbar at 39 °C). The release of the vacuum was made with inert gas (N₂). The obtained product was purified by column chromatography (RP-C18; eluent MeOH/water 7:3) to give the product as a yellow solid. Yield: 35%. ¹H NMR (500 MHz, DMSO-d₆): δ 6.90-6.75 (m, 1H, CH═CH—); 6.69 (s, 1H, Ph-H); 6.64 (dd, 1H, J=16.4, 7.9 Hz, —CH═CH—); 6.44 (dd, 2H, J=24.0, 15.1 Hz, —CH═CH—); 6.20-6.30 (m, 2H, —CH═CH—); 3.75 (s, 3H, —O—CH₃); 3.40-3.35 (in, 4H, CH₂); 3.48 (in, 1H, ═CH); 2.50 (s, 3H, CH₃); 2.25 (s, 3H, CH₃); 2.22 (s, 3H, CH₃); 2.15 (s, 1H, CH); 2.14 (s, 6H, CH₃); 1.75, (m, a (2H) cyclohexyl); 1.65, (m, e (2H) cyclohexyl); 1.60, (m, a (2H) cyclohexyl); 1.58, (m, a (2H) cyclohexyl); 1.25, (m, e (2H) cyclohexyl).

HPLC: Rt 27.03 min, 99.21% (Max); m/z=568.26; 544.37.

### 4.2 Formulation with API

4.2.1 Formulation with Docetaxel

4.5 mg of compound 4 was dispensed into a round bottom flask and dissolved in methanol (3 mL). 0.305 μL of a stock solution of docetaxel in methanol (4.96 mg/mL) was added. The solution was evaporated to dryness in a rotary evaporator. Final drying was performed in a desiccator (40 min) to remove residual solvent. Rehydration of the dried film was performed by adding 0.9% sodium chloride solution (1.5 mL). This solution was then further diluted with 0.9% sodium chloride solution, resulting in yellow transparent solution. The results for different dilutions are shown in the table below.

### Ratio 3:1

| Docetaxel | | |
|---|---|---|
| conc. | Particle | Polydispersity |
| [mg/mL] | size [nm] | index |
| 1 | 8.9 | 0.32 |
| 0.5 | 8.9 | 0.25 |
| 0.25 | 8.7 | 0.31 |

A stock solution of docetaxel in methanol was mixed with a stock solution of compound 4 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of docetaxel. The final weight ratio of docetaxel to micelle forming agent was 1:5. Measurements of particle size and polydispersity index were made over time, as shown in the following table. Increase in the particle size and variations of the polydispersity index over time were noted (see table below).

| Time | Particle | Polydispersity |
|---|---|---|
| [h] | size [nm] | index |
| 0 | 34.56 | 0.26 |
| 1 | 54.37 | 0.14 |
| 2 | 56.46 | 0.12 |
| 4 | 58.90 | 0.11 |
| 6 | 59.79 | 0.12 |
| 24 | 78.72 | 0.08 |

### Example 5

### 5.1 Synthesis of Compound 5

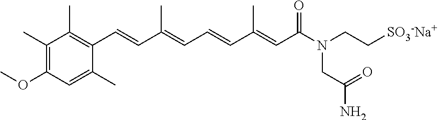

Acitretin (250 mg, 0.77 mmol) was suspended in DMF (2.5 mL) and N-(2-acetamido)-taurine (140 mg, 0.77 mmol) was added followed by DIPEA (400 μL, 2.3 mmol). The yellow suspension was cooled to 15 °C and PyBOP (478 mg, 0.9 mmol) was added. The resulting mixture was stirred at room temperature for 24 hours. The reaction mixture was poured into cold water (10 mL), acidified with 1M HCl to pH 1 and extracted with EtOAc (3×15 mL). As UPLC showed that no product was present in the organic extracts, the organic layer was discarded. The aqueous layer was saturated with solid NaCl. After several minutes a yellow solid precipitated. The suspension was sonicated for 1 minute and then stirred at room temperature for 15 minutes. The solid was filtered off, washed with a small amount of water and dried under vacuum to give 240 mg of the product as a yellow solid (purity 99.9%).

235 mg of solid was dissolved in MeOH (5 mL) and a solution of NaOH (19 mg) in MeOH (0.5 mL) was then added. The solution was stirred for 1 hour and a yellow solid precipitated. The mixture was concentrated to ca. 3 mL, stirred for 30 minutes and then filtered. The solid was washed with a small amount of MeOH and dried. Yield: 41% (160 mg).

¹H NMR (300 MHz, DMSO-d₆): δ 7.51 (d, J=9.5 Hz, 1H), 7.14-6.79 (m, 2H), 6.66 (d, J=19.1 Hz, 2H), 6.45-5.94 (m, 4H), 3.90 (d, J=31.6 Hz, 2H), 3.76 (s, 3H), 3.56 (dt, J=23.1, 7.4 Hz, 2H), 2.66 (dt, J=13.9, 7.0 Hz, 2H), 2.26 (s, 3H), 2.19 (s, 3H), 2.06 (d, J=6.8 Hz, 9H).

m/z=513.19; 491.41.

### 5.2 Formulation with API

5.2.1 Formulation with Docetaxel

A stock solution of docetaxel in methanol was mixed with a stock solution of compound 5 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of docetaxel. The final weight ratio of docetaxel to micelle forming agent was 1:3. Measurements of particle size and polydispersity index were made over time, as shown in the following table. An increase in the particle size and very small variations of the polydispersity index over time were noted.

| Time | Particle | Polydispersity |
|---|---|---|
| [h] | size [nm] | index |
| 0 | 59.40 | 0.17 |
| 1 | 66.24 | 0.16 |
| 2 | 75.40 | 0.14 |
| 4 | 82.19 | 0.13 |
| 6 | 87.95 | 0.13 |
| 24 | 105.7 | 0.14 |

5.2.2 Formulation with Cabazitaxel

A stock solution of cabazitaxel in methanol was mixed with a stock solution of compound 5 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of cabazitaxel. The resulting solution was filtered through a 0.22 μm sterile filter and further diluted with 0.9% sodium chloride solution to 0.5 mg/mL of cabazitaxel. The final weight ratio of cabazitaxel to micelle forming agent was 1:3. Measurements of particle size and polydispersity index were made over time, as shown in the following table. Small variations in the particle size and the polydispersity index over time were noted (see table below).

| Time | Particle | Polydispersity |
|---|---|---|
| [h] | size [nm] | index |
| 0 | 93.83 | 0.19 |
| 1 | 98.08 | 0.20 |
| 2 | 102.2 | 0.21 |
| 4 | 106.6 | 0.20 |
| 6 | 108.8 | 0.21 |
| 24 | 83.42 | 0.39 |

### Example 6

### 6.1 Synthesis of Compound 6

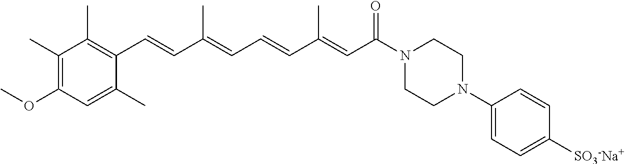

Acitretin (300 mg, 0.92 mmol) was suspended in DMF (3 mL) and DIPEA (380 μL, 2.9 mmol) was added followed by PyBOP (622 mg, 1.2 mmol). The red solution was stirred for 15 minutes at room temperature and 4-(piperazin-1-yl)benzene-1-sulfonic acid (223 mg, 0.92 mmol) was added. The resulting mixture was stirred at room temperature for 24 hours. UPLC showed 93% conversion to the product. The reaction mixture was poured into cold water (20 mL), acidified with 1M HCl to pH 1 and treated with EtOAc, which resulted in the precipitation of an oily solid. The mixture was then saturated with solid NaCl. The organic solvent was evaporated and the residual aqueous suspension stirred at room temperature for 20 minutes. The precipitated solid was filtered off, washed twice with water and dried under vacuum to give 575 mg of solid (purity>91%). The crude product was treated with EtOAc (15 mL) for 1 hour at room temperature and the mixture was then centrifuged. The solvent was decanted, the residue was washed with EtOAc and again centrifuged. The contents were poured into a flask and dried to give 365 mg of beige solid (purity>98%). 360 mg of the solid was suspended in MeOH (20 mL) and a solution of NaOH (49 mg) in MeOH (1.5 mL) was then added, giving a clear yellow solution. The solution was concentrated and the residue was triturated with MeOH (ca. 8 mL) at room temperature for 1.5 hours. The precipitated solid was filtered off, washed with MeOH and dried under vacuum (purity 99.5%). Yield: 21% (110 mg).

¹H NMR (300 MHz, DMSO-d₆): δ 7.49-7.41 (m, 2H), 6.96-6.82 (m, 3H), 6.67 (d, J=17.1 Hz, 2H), 6.47 (d, J=15.1 Hz, 1H), 6.28 (dd, J=15.8, 3.6 Hz, 3H), 3.76 (s, 3H), 3.69-3.55 (m, 4H), 3.22-3.09 (m, 4H), 2.26 (s, 3H), 2.19 (s, 3H), 2.06 (d, J=3.4 Hz, 9H).

m/z (M−23)=549.24.

### 6.2 Formulation with API

6.2.1 Formulation with Docetaxel

A stock solution of docetaxel in methanol was mixed with a stock solution of compound 6 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of docetaxel. The final weight ratio of docetaxel to micelle forming agent was 1:3. Measurements of particle size and polydispersity index were made over time. Small variations in the particle size and the polydispersity index over time were noted (see table below).

| Time | Particle | Polydispersity |
|---|---|---|
| [h] | size [nm] | index |
| 0 | 61.37 | 0.22 |
| 1 | 61.27 | 0.28 |
| 2 | 64.21 | 0.28 |
| 4 | 67.34 | 0.28 |
| 6 | 69.84 | 0.28 |
| 24 | 66.18 | 0.38 |

### Example 7

### 7.1 Synthesis of Compound 7

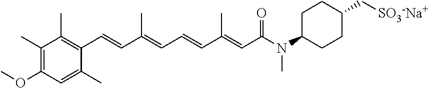

Acitretin (250 mg, 0.8 mmol) was suspended in anhydrous DMF (5 mL). DIPEA (297 mg, 2.3 mmol) was then added followed by PyBOP (518 mg, 1.0 mmol). The reaction was stirred at room temperature, under an argon atmosphere, for 10 minutes and trans-(4-methylamino)cyclohexyl)methanesulfonic acid (159 mg, 0.8 mmol) was then added in one portion. The progress of the reaction was followed by UPLC (91% conversion was achieved after 24 hours).

The reaction mixture was poured into cold water (15 mL) and acidified with 1M HCl to pH 1. Extraction with ethyl acetate was attempted but the phases did not separate. The organic solvent was then evaporated and solid NaCl was added until the mixture was saturated. The formed solid was filtered off, dried and purified by column chromatography (FC, RP-C18, 30-70% MeOH in water in 35 minutes). Fractions containing the product were concentrated and then dissolved in 5 mL of MeOH containing 1.0 eq. of NaOH. The mixture was stirred at room temperature for 1 hour and then evaporated to dryness. The obtained powder was triturated with acetone and then dried under vacuum (0.2 mbar) at room temperature for 24 hours. In this manner, 180 mg of yellow powder (with purity 99.5%) was obtained.

¹H NMR (300 MHz, DMSO-d₆): δ 6.92-6.75 (m, 1H), 6.72-6.58 (m, 2H), 6.53-6.36 (m, 1H), 6.34-6.01 (m, 2H), 4.21 (d, J=8.4 Hz, 1H), 3.76 (s, 3H), 3.55 (d, J=18.2 Hz, 1H), 2.78 (d, J=21.5 Hz, 3H), 2.31 (dd, J=8.2, 6.0 Hz, 2H), 2.26 (s, 3H), 2.19 (s, 3H), 2.10-2.04 (m, 6H), 1.99 (d, J=8.2 Hz, 3H), 1.52 (d, J=20.0 Hz, 5H), 1.00 (s, 2H).

m/z (M−23)=514.23.

### 7.2 Formulation with API

7.2.1 Formulation with Docetaxel

A stock solution of docetaxel in methanol was mixed with a stock solution of compound 7 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of docetaxel. The final weight ratio of Docetaxel to micelle forming agent was 1:3. Measurements of particle size and polydispersity index were made over time, as shown in the following table. Small variations in the particle size and the polydispersity index over time were noted (see table below).

| Time | Particle | Polydispersity |
|---|---|---|
| [h] | size [nm] | index |
| 0 | 100.8 | 0.23 |
| 1 | 97.60 | 0.24 |
| 2 | 97.60 | 0.24 |
| 4 | 96.90 | 0.25 |
| 6 | 99.12 | 0.25 |
| 24 | 96.76 | 0.26 |

### Example 8

### 8.1 Synthesis of Compound 8

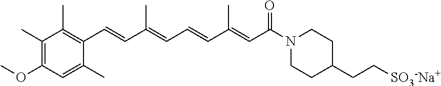

Acitretin (250 mg, 0.8 mmol) was suspended in anhydrous DMF (5 mL). DIPEA (297 mg, 2.3 mmol) was then added followed by PyBOP (518 mg, 1.0 mmol). The reaction was stirred at room temperature, under an argon atmosphere, for 10 minutes. 2-(Piperidin-4-yl)ethane-1-sulfonic acid (148 mg, 0.8 mmol) was then added in one portion. The progress of the reaction was followed by UPLC.

The reaction mixture was poured into cold water (15 mL) and acidified with 1M HCl to pH 1. The mixture was saturated with solid NaCl, but no solid was formed. The solution was purified by column chromatography (FC, RP-C18, 30-70% MeOH in water in 35 minutes).

Fractions containing the product were concentrated and then dissolved in 5 mL of MeOH containing 1.0 eq. of NaOH. The mixture was stirred at room temperature for 1 hour and then evaporated to dryness. The obtained powder was triturated with acetone and then dried under vacuum (0.2 mbar) at room temperature for 24 hours. In this manner, 190 mg of yellow powder (purity 99.7%) was obtained.

¹H NMR (300 MHz, DMSO-d₆): δ 6.83 (dd, J=15.2, 11.3 Hz, 1H), 6.74-6.60 (m, 2H), 6.43 (d, J=15.1 Hz, 1H), 6.33-6.11 (m, 3H), 4.38 (d, J=12.9 Hz, 1H), 3.83 (d, J=13.6 Hz, 1H), 3.76 (s, 3H), 2.96 (t, J=12.9 Hz, 1H), 2.46-2.35 (m, 2H), 2.26 (s, 3H), 2.19 (s, 3H), 2.08-2.02 (m, 7H), 1.99 (d, J=1.0 Hz, 3H), 1.67 (d, J=12.8 Hz, 2H), 1.60-1.45 (m, 3H), 1.07-0.83 (m, 3H).

m/z=502.19; 500.21.

### 8.2 Formulation with API

8.2.1 Formulation with Docetaxel

A stock solution of docetaxel in methanol was mixed with a stock solution of compound 8 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of docetaxel. The final weight ratio of Docetaxel to micelle forming agent was 1:3. Measurements of particle size and polydispersity index were made over time, as shown in the following table. Increase in the particle size and small variations the polydispersity index over time were noted (see table below).

| Time | Particle | Polydispersity |
|---|---|---|
| [h] | size [nm] | index |
| 0 | 55.38 | 0.20 |
| 1 | 70.34 | 0.15 |
| 2 | 75.90 | 0.13 |
| 4 | 83.10 | 0.11 |
| 6 | 87.94 | 0.12 |
| 24 | 105.0 | 0.16 |

### Example 9

### 9.1 Synthesis of Compound 9

### Step 1

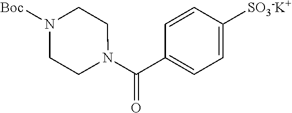

Boc-piperazine (1.0 g, 5.37 mmol) was dissolved in anhydrous DMF (10 mL) under inert atmosphere. PyBop (3.63 g, 7.0 mmol) and DIPEA (2.81 mL, 16.1 mmol, 3.0 eq.) were then added and the reaction mixture was stirred for 5 minutes at room temperature. 4-sulfo-benzoic acid potassium salt (1.29 g, 5.37 mmol) was then added in one portion. The reaction mixture was stirred at room temperature overnight. The precipitated product was filtered off and dried. Yield: 76%.

### Step 2

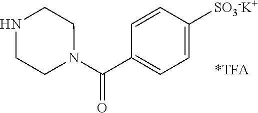

The compound of step 1 (0.5 g, 1.22 mmol) was added to a mixture of TFA and DCM (1:1, 5 mL), and the reaction mixture was stirred at room temperature for 30 minutes. The solvent was then evaporated. MeOH (5 mL) was added and after a while a white solid precipitated. The solid was filtered off and washed with small amount of MeOH. Yield: 0.217 g (42%); purity 97%.

### Step 3

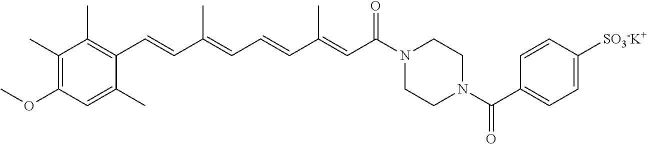

Acitretin (100 mg, 0.31 mmol) was suspended in anhydrous DMF (2 mL) under an argon atmosphere and DIPEA (215 μL, 1.2 mmol) and PyBop (208 mg, 0.4 mmol) were added. The reaction mixture was stirred at room temperature for 3 minutes and the compound of step 2 (130 mg, 0.3 mmol) was then added in one portion. The solution was stirred overnight at room temperature. The product was isolated via chromatography column (RP-C18, water/MeOH). The residue was triturated with acetone to give the product as a yellow powder (purity 99.4%). Yield: 24 mg (13%).

¹H NMR (300 MHz, DMSO-d₆): δ 7.66 (d, J=8.1 Hz, 2H), 7.48-7.31 (m, 2H), 6.89 (dd, J=15.3, 11.4 Hz, 1H), 6.67 (d, J=16.9 Hz, 2H), 6.43 (d, J=15.1 Hz, 1H), 6.26 (t, J=14.9 Hz, 2H), 3.76 (s, 3H), 3.55 (s, 8H), 2.26 (s, 3H), 2.19 (s, 3H), 2.10-2.03 (m, 9H).

m/z=579.29; 577.19.

### 9.2 Formulation with API

9.2.1 Formulation with Docetaxel

A stock solution of docetaxel in methanol was mixed with a stock solution of compound 9 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of docetaxel. The final weight ratio of docetaxel to micelle forming agent was 1:3. Measurements of particle size and polydispersity index were made over time, as shown in the following table. Increase in the particle size and small variations the polydispersity index over time were noted (see table below).

| Time | Particle | Polydispersity |
|---|---|---|
| [h] | size [nm] | index |
| 0 | 89.91 | 0.20 |
| 1 | 97.93 | 0.20 |
| 2 | 103.8 | 0.20 |
| 4 | 113.0 | 0.17 |
| 6 | 118.6 | 0.17 |
| 24 | 122.0 | 0.22 |

### Example 10

### 10.1 Synthesis of Compound 10

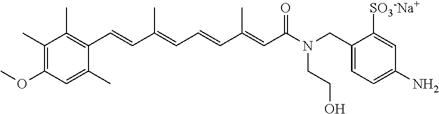

Acitretin (400 mg, 1.23 mmol) was suspended in DMF (4 mL) and DIPEA (640 μL, 3.7 mmol) was added followed by PyBOP (765 mg, 1.5 mmol, 1.2 eq.). The solution was stirred for 5 minutes at room temperature and 5-amino-2-[(2-hydroxy-ethylamino)-methyl]-benzenesulfonic acid (302 mg, 1.23 mmol) was then added. The resulting mixture was stirred at room temperature for 21 hours. UPLC showed 86% conversion to product. The reaction mixture was poured into cold water (40 mL) and acidified with 1M HCl to pH 1. The mixture was saturated with NaCl, and a gummy solid was formed. The mixture was sonicated for 20 minutes and then stirred at room temperature for 30 minutes. The precipitated solid was centrifuged. The solvent was decanted, the residue was washed with water (15 mL) and centrifuged again. The procedure of washing and centrifugation was then repeated 2 times. The contents were poured into a flask and dried. The solid was triturated with EtOAc (25 mL) at room temperature for 1 hour and then centrifuged. The solvent was decanted, the residue was washed with EtOAc (15 mL) and centrifuged again. The procedure of washing and centrifugation was then repeated 2 times. The residue was then dried to give 560 mg of solid (purity 84%). 555 mg of solid was suspended in MeOH (50 mL). A solution of NaOH (75 mg) in MeOH (1.5 mL) was added, giving a clear solution. The solution was stirred for 10 minutes at room temperature and concentrated. The brown foam that was obtained was dissolved in MeOH (5 mL) and purified by column chromatography (C18; eluent MeOH/water 3:7 to 7:3) to obtain the product (purity 99.5%). Yield: 110 mg (21%).

¹H NMR (300 MHz, DMSO-d₆) δ 7.08 (dd, J=4.2, 2.5 Hz, 1H), 6.93-6.58 (m, 4H), 6.52-6.36 (m, 2H), 6.34-6.06 (m, 3H), 5.02 (d, J=15.6 Hz, 2H), 4.89 (d, J=9.4 Hz, 2H), 4.66 (d, J=15.3 Hz, 1H), 3.75 (d, J=2.7 Hz, 3H), 3.58-3.36 (m, 3H), 3.25 (t, J=6.3 Hz, 1H), 2.25 (d, J=6.4 Hz, 3H), 2.18 (d, J=6.8 Hz, 3H), 2.14-1.99 (m, 9H).

m/z (M-Na)=533.18; 554.93.

### 10.2 Formulation with API

10.2.1 Formulation with Docetaxel

A stock solution of docetaxel in methanol was mixed with a stock solution of compound 10 in a round bottom flask. The mixture was evaporated to dryness in a rotary evaporator. Final drying was performed in a desiccator to remove residual solvent. Hydration of the dried film was performed by adding water for injection (1.0 mL) to a concentration of 1 mg/mL of docetaxel. The resulting solution was filtered through a 0.22 μm sterile filter and further diluted with 0.9% sodium chloride solution to 0.5 mg/mL of Docetaxel. The final weight ratio of docetaxel to micelle forming agent was 1:5. Measurements of particle size and polydispersity index were made over time, as shown in the following table. Increase in the particle size and minor decrease the polydispersity index over time were noted (see table below).

| Time | Particle | Polydispersity |
|---|---|---|
| [h] | size [nm] | index |
| 0 | 52.29 | 0.16 |
| 1 | 62.51 | 0.13 |
| 2 | 70.07 | 0.12 |
| 4 | 78.98 | 0.12 |
| 6 | 85.10 | 0.10 |
| 24 | 107.4 | 0.10 |

1. A compound of formula (I) 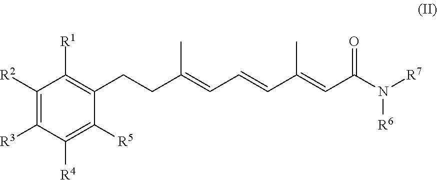
wherein
R¹, R², R³, R⁴ and R⁵ are each independently selected from the group consisting of hydrogen, halogen, hydroxy, C₁₋₆ alkyl, C₁₋₆ haloalkyl, C₁₋₆ alkoxy or C₁₋₄ alkoxycarbonyl;
R⁶ is selected from the group consisting of halogen, C₁₋₄ alkyl, C₁₋₄ hydroxyalkyl, C₃₋₈ cycloalkyl, C₁₋₄ alkylcarbonyl, C₂₋₄ alkenylcarbonyl, C₃₋₈ cycloalkylcarbonyl, aminocarbonyl-C₁₋₄ alkyl, —C(═NH)NH₂, and phenyl, wherein phenyl is optionally substituted with one or more substituents selected from the group consisting of halogen, hydroxy, C₁₋₄ alkyl, C₁₋₄ alkoxy and amino;
R⁷ is —(CR^{8A}R^{8B})ₙ—X, benzyl-X or C₃₋₇-cycloalkyl-(CR^{8A}R^{8B})ₘ—X, wherein n is an integer 2 or 3 and m is an integer 1 or 2, and wherein benzyl is optionally additionally substituted with one or more substituents selected from the group consisting of halogen, hydroxy, C₁₋₄ alkyl, C₁₋₄ alkoxy and amino;
or R⁶ and R⁷, together with the nitrogen atom to which they are attached, form a 5- to 7-membered saturated heterocyclic ring, which is substituted with —(CR^{8A}R^{8B})ₚ—X, phenyl-X or benzoyl-X, wherein p is an integer 1 or 2, and wherein phenyl or benzoyl is optionally additionally substituted with one or more substituents selected from the group consisting of halogen, hydroxy, C₁₋₄ alkyl, C₁₋₄ alkoxy and amino;
R^{8A} and R^{8B} are each independently selected from the group consisting of hydrogen, hydroxy, C₁₋₄ alkyl, C₁₋₄ alkoxy, —S(═O)₂OH, —S(═O)OH and —P(═O)(OH)₂; and
X is —S(═O)₂OH, —S(═O)OH or —P(═O)(OH)₂;
or a pharmaceutically acceptable salt thereof.

2. The compound according to claim 1, wherein R¹, R², R³, R⁴ and R⁵ are each independently selected from the group consisting of hydrogen, hydroxy, methyl and methoxy.

3. The compound according to claim 1 or 2, wherein R¹, R² and R⁵ are each methyl, R³ is methoxy and R⁴ is hydrogen.

4. The compound according to any one of claims 1 to 3, wherein R⁶ is methyl, 2-hydroxyethyl, cyclohexyl, prop-2-enoyl, 1,1-bis(hydroxymethyl)-2-hydroxyethyl or aminocarbonylmethyl.

5. The compound according to any one of claims 1 to 4, wherein R⁷ is —(CR^{8A}R^{8B})ₙ—X, wherein n is an integer 2 or 3 and wherein R^{8A} and R^{8B} are each independently selected from the group consisting of hydrogen and hydroxy.

6. The compound according to any one of claims 1 to 4, wherein R⁷ is benzyl-X, and wherein benzyl is additionally substituted with amino.

7. The compound according to any one of claims 1 to 4, wherein R⁷ is cyclohexyl-methyl-X.

8. The compound according to any one of claims 1 to 4, wherein R⁶ and R⁷, together with the nitrogen atom to which they are attached, form a piperidine or piperazine ring, which is substituted with ethyl-X, phenyl-X or benzoyl-X.

9. The compound according to any one of claims 1 to 8, wherein X is —S(═O)₂OH.

10. The compound according to claim 1, selected from the group consisting of: 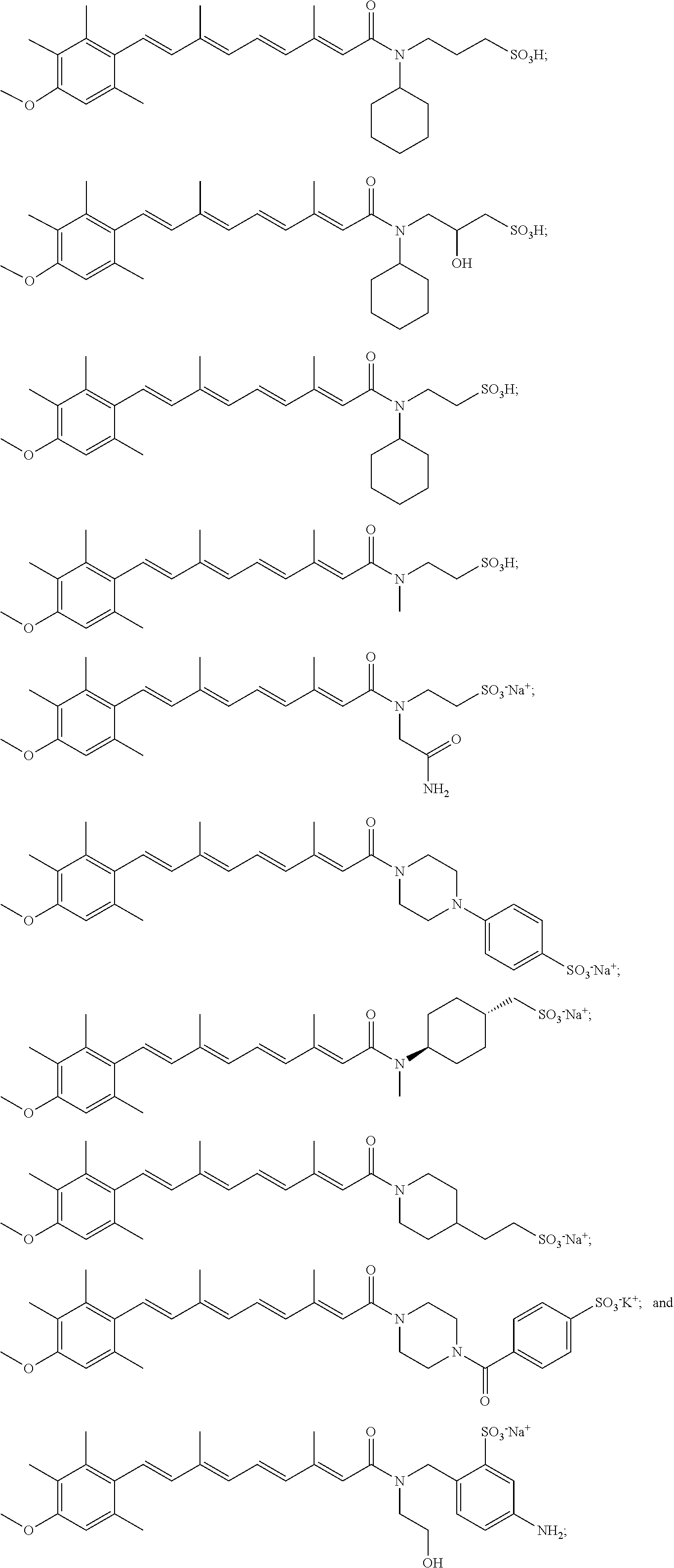
or a pharmaceutically acceptable salt thereof.

11. A pharmaceutical composition comprising at least one compound according to any one of claims 1 to 10.

12. The pharmaceutical composition according to claim 11, further comprising an active pharmaceutical ingredient.

13. The pharmaceutical composition according to claim 12, wherein the active pharmaceutical ingredient is a hydrophobic drug.

14. The pharmaceutical composition according to claim 12, wherein the active pharmaceutical ingredient is a cytotoxic drug, preferably selected from the group consisting of docetaxel, paclitaxel, cabazitaxel, doxorubicin and mitoxantrone.

15. The pharmaceutical composition according to any one of claims 12 to 14, wherein the at least one compound according to any one of claims 1 to 10 and the active pharmaceutical ingredient form a micelle.

16. A drug micelle, comprising at least one compound according to any one of claims 1 to 10 and a drug.

17. The drug micelle according to claim 16, wherein the drug is a cytotoxic drug, preferably selected from the group consisting of docetaxel, paclitaxel, cabazitaxel, doxorubicin and mitoxantrone.

18. The pharmaceutical composition according to claim 14 or the drug micelle according to claim 17, for use in the treatment of cancer.

19. A method for potentiating the efficacy of a pharmaceutically active substance, wherein said substance is prepared in micellar form with a compound according to any one of claims 1 to 10, or a pharmaceutically acceptable salt thereof.

20. A method for increasing the solubility of a pharmaceutically active substance, wherein said substance is prepared in micellar form with a compound according to any one of claims 1 to 10, or a pharmaceutically acceptable salt thereof.

21. A method for improving the pharmacokinetic or pharmacodynamic properties of a pharmaceutically active substance, wherein said substance is prepared in micellar form with a compound according to any one of claims 1 to 10, or a pharmaceutically acceptable salt thereof.

22. A method for improving the storage properties of a pharmaceutically active substance, wherein said substance is prepared in micellar form with a compound according to any one of claims 1 to 10, or a pharmaceutically acceptable salt thereof.
